(19) United States
(12) Patent Application Publication
(10) Pub. No.: US 2022/0257233 A1
(43) Pub. Date: Aug. 18, 2022

(54) CONTINUOUS TETHERED TISSUE ANCHOR AND ASSOCIATED SYSTEMS AND METHODS

(71) Applicant: W. L. Gore & Associates, Inc., Newark, DE (US)

(72) Inventor: Joshua C. Haarer, Flagstaff, AZ (US)

(21) Appl. No.: 17/619,452

(22) PCT Filed: May 8, 2020
(86) PCT No.: PCT/US2020/032054

§ 371 Date: Dec. 15, 2021

Related U.S. Application Data
(60) Provisional application No. 62/965,610, filed on Jan. 24, 2020.
(60) Provisional application No. 62/862,519, filed on Jun. 17, 2019.

(51) Int. Cl. A61B 17/04; A61B 17/00

(52) U.S. Cl. CPC A61B 17/0401; A61B 17/00234; A61B 2017/00243; A61B 2017/0464; A61B 2017/0406; A61B 2017/0437; A61B 2017/00526

(57) ABSTRACT

The invention relates to tissue anchor in medical procedures. More specifically the invention relates to a tethered anchor. Tethered anchor including a tether portion and an anchor portion that are continuously braided with one another, the tether portion extending continuously from the anchor portion; and the anchor portion having a greater width than a width of the tether portion.

### CROSS-REFERENCE TO RELATED APPLICATION

This application is a national phase application of PCT Application No. PCT/US2020/032054, internationally filed on May 8, 2020, which claims the benefit of Provisional Application No. 62/965,610, filed Jan. 24, 2020, and also claims the benefit of Provisional Application No. 62/862,519, filed Jun. 17, 2019, which are incorporated herein by reference in their entireties for all purposes.

### BACKGROUND

Various tissue anchors have been proposed for delivery into a body of a patient. In some examples, such anchors are delivered using a percutaneous or transcatheter approach (e.g., using a delivery catheter). Some tissue anchors have been proposed that utilize a pledget and tether arrangement. Such anchors may be utilized to secure anatomical structures to one another, to secure an anatomical structure to an implanted device, or to secure two implanted devices together, for example.

### SUMMARY

Various disclosed concepts relate to continuous, or integral implantable tissue anchors including an anchor portion and a tether portion that are seamlessly interconnected. Some examples relate to braided anchor and tether portions, where the anchor portion and/or tether portion has one or more sections in which the braiding pattern varies to exhibit enhanced physical properties according to expected use.

According to one example (“Example 1”), a tethered anchor includes a tether portion and an anchor portion. The tether portion and the anchor portion are configured to be continuously braided with one another. The tether portion being elongate and extending continuously from the anchor portion. The anchor portion having a greater width than a width of the tether portion when the anchor portion is collapsed.

According to another example further to Example 1 (“Example 2”), the tether portion defines a rounded transverse profile and the anchor portion defines a flat transverse profile.

According to another example further to Example 1 (“Example 3”), the tether portion defines a flat transverse profile and the anchor portion defines a rounded transverse profile.

According to another example further to Example 1 (“Example 4”), the tether portion and the anchor portion both define a rounded transverse profile.

According to another example further to Example 1 (“Example 5”), the tether portion and the anchor portion both define a flat transverse profile.

According to another example further to Example 1 (“Example 6”), the anchor portion defines a plurality of crossing apertures, and further wherein a length of the tether portion is passed through the crossing apertures of the anchor portion.

According to another example further to Example 1 (“Example 7”), the tether portion is characterized by a first pick count and the anchor portion is characterized by a second pick count that is different than the first pick count.

According to another example further to Example 1 (“Example 8”), a pick count of the tether portion and anchor portion varies.

According to another example further to Example 1 (“Example 9”), a section of the tether portion is characterized by a first pick count and another section of the tether portion is characterized by a second pick count that is greater than the first pick count.

According to another example further to Example 1 (“Example 10”), the tethered anchor further includes at least part of the anchor portion is characterized by a braid pattern configured to encourage tissue ingrowth.

According to another example further to Example 1 (“Example 11”), the anchor portion comprises a plurality of individual braid filaments and the anchor portion is thermally treated to bond the individual braid filaments together.

According to another example further to Example 1 (“Example 12”), the tethered anchor includes a stop component coupled to the tether portion and configured to prevent the tether portion from passing through the anchor portion.

According to another example further to Example 1 (“Example 13”), at least one of the tether portion and the anchor portion incorporates an elastomeric axial member therein.

According to another example (“Example 14”), a tether anchor prepared by a process of continuously braiding a tether portion and an anchor portion, the tether portion being elongate and extending continuously from the anchor portion, the anchor portion having a greater width than a width of the tether portion.

According to another example further to Example 14 (“Example 15”), the tether portion is braided to define a rounded transverse profile and the anchor portion is braided to define a flat transverse profile.

According to another example further to Example 14 (“Example 16”), the tether portion is braided to define a flat transverse profile and the anchor portion is braided to define a rounded transverse profile.

According to another example further to Example 14 (“Example 17”), the tether portion and the anchor portion are both braided to define a rounded transverse profile.

According to another example further to Example 14 (“Example 18”), the tether portion and the anchor portion are both braided to define a flat transverse profile.

According to another example further to Example 14 (“Example 19”), the anchor portion is braided to define a plurality of crossing apertures, and further wherein a length of the tether portion is passed through the crossing apertures of the anchor portion.

According to another example further to Example 14 (“Example 20”), the tether portion is braided at a first pick count and the anchor portion is braided at a second pick count that is different than the first pick count.

According to another example further to Example 14 (“Example 21”), the tether portion and anchor portion are braided at a varying pick count.

According to another example further to Example 14 (“Example 22”), a section of the tether portion is braided at a first pick count and another section of the tether portion is braided at a second pick count that is greater than the first pick count.

According to another example further to Example 14 (“Example 23”), at least part of the anchor portion is braided at a braid pattern configured to encourage tissue ingrowth.

According to another example (“Example 24”), a method of forming a tethered anchor includes continuously braiding a tether portion and an anchor portion, the tether portion being elongate. The method also includes extending the tether portion continuously from the anchor portion, the anchor portion having a greater width than a width of the tether portion.

According to another example further to Example 24 (“Example 25”), the tether portion is braided to define a rounded transverse profile and the anchor portion is braided to define a flat transverse profile.

According to another example further to Example 24 (“Example 26”), the tether portion is braided to define a flat transverse profile and the anchor portion is braided to define a rounded transverse profile.

According to another example further to Example 24 (“Example 27”), the tether portion and the anchor portion are both braided to define a rounded transverse profile.

According to another example further to Example 24 (“Example 28”), the tether portion and the anchor portion are both braided to define a flat transverse profile.

According to another example further to Example 24 (“Example 29”), the anchor portion is braided to define a plurality of crossing apertures, and further wherein a length of the tether portion is passed through the crossing apertures of the anchor portion.

According to another example further to Example 24 (“Example 30”), the tether portion is braided at a first pick count and the anchor portion is braided at a second pick count that is different than the first pick count.

According to another example further to Example 24 (“Example 31”), the tether portion and anchor portion are braided at a varying pick count.

According to another example further to Example 24 (“Example 32”), a section of the tether portion is braided at a first pick count and another section of the tether portion is braided at a second pick count that is greater than the first pick count.

According to another example further to Example 24 (“Example 33”), at least part of the anchor portion is braided at a braid pattern configured to encourage tissue ingrowth.

According to one example (“Example 34”), a method of treating heart valve disfunction includes arranging a tethered anchor at a target location within a patient, the tethered anchor including a tether portion and an anchor portion that are continuously braided with one another with the tether portion being elongate and extending continuously from the anchor portion and with anchor portion having a greater width than a width of the tether portion.

According to another example (“Example 35”), further to the method of Example 34, the tethered anchor is configured for chordal repair or replacement or treating a defective valve.

According to one example (“Example 36”) a tethered anchor includes a tether portion and at least one anchor portion that are continuously braided with one another, the tether portion being elongate and extending continuously from the at least one anchor portion and the at least one anchor portion having a greater width than a width of the tether portion; and at least one surgical needle coupled to the at least one anchor portion.

According to another example (“Example 37”), further to the tethered anchor of Example 36, the at least one anchor portion includes a first anchor portion arranged at an end of the tether portion and a second anchor portion arranged at another end of the tether portion.

According to another example (“Example 38”), further to the tethered anchor of Example 37, at least one surgical needle includes a first surgical needle coupled to the first anchor portion and a second surgical needle coupled to the second anchor portion.

According to one example (“Example 39”), a tethered anchor includes a tether portion and at least one anchor portion that are continuously braided with one another, the tether portion being elongate and extending continuously from the at least one anchor portion and the at least one anchor portion having a greater width than a width of the tether portion; and at least one tissue anchor coupled to the at least one anchor portion.

According to another example (“Example 40”), further to the tethered anchor of Example 39, the at least one anchor portion includes a first anchor portion arranged at an end of the tether portion and a second anchor portion arranged at another end of the tether portion.

According to another example (“Example 41”), further to the tethered anchor of Example 39, wherein at least one tissue anchor includes a first tissue anchor coupled to the first anchor portion and a second tissue anchor coupled to the second anchor portion.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a braided pledget anchor and suture according to some embodiments;

FIG. 2 shows the braided pledget anchor and suture of FIG. 1 in a partially actuated configuration;

FIG. 3 shows the braided pledget anchor and suture of FIGS. 1 and 2 in a fully actuated configuration;

FIG. 4 shows a portion of a braided pledget anchor and suture according to one embodiment to show the details of the braidings;

FIG. 5 shows a braided pledget anchor and suture according to some embodiments;

FIG. 6 shows a braided pledget anchor and suture according to some embodiments;

FIG. 7 shows a braided pledget anchor and suture according to some embodiments;

FIG. 8 shows a braided pledget anchor and suture according to some embodiments;

FIG. 9 shows an anchor portion of a tethered anchor having a surgical needle, according to some embodiments; and

FIG. 10 shows a tethered anchor and surgical needle as used in chordal repair procedures according to some embodiments;

FIG. 11 shows end portions of a tethered anchor and surgical needles arranged at each of the ends according to some embodiments;

FIG. 12 shows an end portion of a tethered anchor and a tissue anchor arranged at the end portion according to some embodiments; and

FIG. 13 shows an end portion of a tethered anchor and a tissue anchor arranged at the end portion according to some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example.

As used herein, “couple” means to join, connect, attach, adhere, affix, or bond, whether directly or indirectly, and whether permanently or temporarily.

The term “pick count” as used herein refers to a number of strand crossings per unit length such as per inch or per centimeter of a braided structure. The higher the number of pick count, the finer the braided structure is.

The term “braid angle” as used herein refers to an acute angle measured from the axis of the braid to the axis of the material forming the braid, thereby defining the orientation of the material forming the braid.

The term “aspect ratio” as used herein refers to a ratio of a width of a transverse cross-section to the height of the transverse cross-section.

### Description of Various Embodiments

Various disclosed concepts relate to continuous, or integral implantable anchors including an anchor portion and a tether portion that are seamlessly interconnected. Some examples relate to braided anchor and tether portions, where the anchor portion and/or tether portion has one or more sections in which the braiding pattern varies (e.g., pick count) to exhibit enhanced physical properties according to expected use.

As described in further detail below, the implantable anchors discussed herein may be used in a variety of medical procedures. For example and in certain instances, the implantable anchors may be used in chordal repair. In addition, the implantable anchors may be used for treating a defective valve (e.g., mitral valve, tricuspid valve). The implantable anchors may be wrapped about a circumference of the heart or valve annulus to ensure closure of a valve that is experiencing regurgitation. In addition, the implantable anchors may be used in valve annuloplasty procedures in a heart or closing an opening or aperture formed in a wall of the heart, such as a ventricular or atrial septal wall defect.

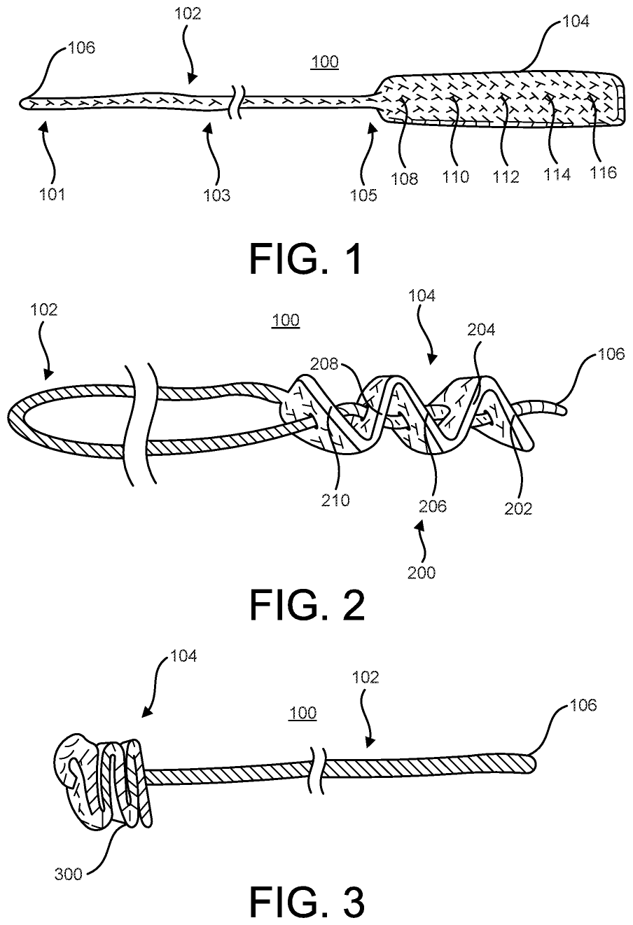

FIG. 1 shows a tethered anchor 100 with a tether portion 102, or suture, and an anchor portion 104, or pledget, where the tether portion 102 and the anchor portion 104 are continuously braided with one another using one or more filaments in a seamless, integral configuration. In various examples, the tethered anchor 100 is formed by continuously braiding (e.g., from one end to another end) one or more filaments together into a tether portion 102 and an anchor portion 104, where the anchor portion 104 has a greater width than a width of the tether portion 102. In some examples, the braid pattern transitions from a tubular braid pattern in the tether portion 102 to a flat braid pattern in the anchor portion 104. In some examples, the tether portion 102 and/or the anchor portion 104 incorporates an axial member (not shown) therein which may be an axial strand made of an elastomeric material such that the axial member provides the tether portion 102 and/or the anchor portion 104 with elastic properties.

In various embodiments, the tether portion 102 is elongate and extends continuously from the anchor portion 104 with the same filament(s) forming the tether portion 102 also forming the anchor portion 104. The tether portion 102 optionally defines an end portion 106 (e.g., a free end), or end portion 106, opposite to the anchor portion 104 and defines a length therebetween. The tether portion 102 may have any of a variety of lengths, such as 1 cm, 10 cm, 100 cm, 200 cm, any value or range between any of the foregoing examples, or more than 200 cm. The tether portion 102 may define any of a variety of diameters, such as 0.1 mm, 0.3 mm, 0.5 mm, 1 mm, 1.5 mm, 2 mm, any value or range between any of the foregoing examples, or more than 2 mm. The tether portion 102 optionally has a round, or tubular transverse cross-section. In some examples, the tether portion 102 includes a transverse cross-section, or transverse outer profile, that defines an aspect ratio of about 1 (i.e., 1:1), or from 0.5 to 2, for example, although a variety of values are also contemplated. In some examples, portions of the tethered anchor 100 may be thermally treated such that the individual filaments forming the anchor portion 104 and/or the tether portion 102 bond to one another. In other instances, an adhesive may be used to bond the portions of the tethered anchor 100 together. A desired length, diameter, and/or cross-section may be selected based on the use for the tethered anchor 100 (e.g. chordal repair, valve procedures, or valve annuloplasty, closing an opening or aperture formed in a wall of the heart, such as a ventricular or atrial septal wall defect).

The anchor portion 104 is optionally flat and tab-like, having a generally rectangular profile from a plan view. The anchor portion 104 may define a relatively high aspect ratio, such as about 5 (i.e., 5:1) or more, 10 or more, 15 or more, or 20 or more, for example. Thus, in some examples, the tether portion 102 optionally defines a round transverse cross-section, or transverse outer profile and aspect ratio approaching 1, while the anchor portion 104 defines a flat transverse cross-section, or transverse outer profile and aspect ratio that is relatively higher than that of the tether portion 102. The anchor portion 104 optionally has one or more crossing apertures 108, 110, 112, 114, 116, such as a plurality of crossing apertures 108, 110, 112, 114, 116 through which the tether portion 102 may pass in an alternating, zig-zag or laced pattern as shown in FIG. 2. The crossing apertures 108, 110, 112, 114, 116 may be formed by gaps or spaces between adjacent filaments of the braid pattern used to form the anchor portion 104 and/or may be otherwise formed through the anchor portion 104.

In one example, the one or more crossing apertures 108, 110, 112, 114, 116 are pre-formed into the anchor portion 104 during manufacture (e.g., during braiding) such that the tether portion 102 only needs to be guided through said crossing aperture(s) 107 to create an arrangement in which the end portion 106 may be tensioned to collapse the anchor portion 104 to transfer the anchor portion 104 from a delivery configuration to an anchoring configuration. In some examples, the anchor portion 104 is formed without any pre-formed crossing aperture or opening, and the crossing apertures are formed after the anchor portion 104 is formed (e.g., braided) by puncturing the surface of the anchor portion 104 to provide the openings through which the tether portion 102 can pass through.

Either as part of manufacture, or prior to or during implantation, a needle or other implement can be utilized to deliver end portion 106 through the crossing apertures 108, 110, 112, 114, 116 and/or to form the crossing apertures 108, 110, 112, 114, 116. The size of the crossing apertures 108, 110, 112, 114, 116 can be the same as, smaller than, or larger than the thickness of the tether portion 102. In some examples, the crossing apertures 108, 110, 112, 114, 116 are smaller than the thickness of the tether portion 102, but the material of the anchor portion 104 is expandable or elastic such that the tether portion 102 can pass through the crossing apertures without causing damage to either the tether portion 102 or the anchor portion 104. The crossing apertures 108, 110, 112, 114, 116 can be evenly spaced apart or have varied spacing. In one example, the crossing apertures are positioned along a relatively straight line, although the crossing apertures 108, 110, 112, 114, 116 may be staggered or otherwise arranged. And, although FIG. 2 shows five crossing apertures, it is understood that any suitable number of crossing apertures can be used.

In some examples, the tether portion defines a proximal section 101 extending from the end portion 106, a distal section 105 adjacent the anchor portion 104, and an intermediate section 103 between the proximal section 101 and the distal section 105. The proximal section 101, intermediate section 103, and the distal section 105 may make up any of a variety of percentages of the length of the tether portion 102, such as ⅓-⅓-⅓, 80%-10%-10%, or any of a variety of combinations. In some examples, each of the sections 101, 103, 105 makes up at least 5% of the length of the tether portion 102. In some examples, a braid pattern used to form the tether portion 102 varies along the length of the tether portion 102. For example, the proximal section 101 may have a first pick count, the intermediate section 103 may have a second pick count, and the distal section 105 may have a third pick count different from each of the first and second pick counts or the same as one of the first and second pick counts.

In some embodiments, at least part of the tether portion 102 (e.g., the proximal section 101, intermediate section 103, and/or distal section 105) is characterized by a pick count that is greater than that of at least part of the anchor portion 104. And, similar to the tether portion 102, the anchor portion 104 optionally varies in braid pattern along its length (e.g., continuously, in a step-wise fashion, or combinations thereof). If desired, the pick count of at least part of the tether portion 102 is less than the pick count of at least part of the anchor portion 104. In yet another example, the pick count is the same throughout the tether portion 102 and the anchor portion 104. In yet another example, the pick count varies in the tether portion 102 and/or the anchor portion 104.

In one example, at least part of the anchor portion 104 (or tether portion 102) is characterized by a braid pattern configured to encourage tissue ingrowth when placed inside a body. In a related example, the entire anchor portion 104 (or tether portion 102) is characterized by a braid pattern configured to encourage tissue ingrowth. Some exemplary braid patterns are characterized by interstitial distances that define pores with at least 75 pm in diameter to encourage tissue ingrowth. In addition, at least part of the anchor portion 104 or tether portion 102 may not encourage tissue ingrowth when placed inside a body.

FIG. 2 shows an intermediate configuration of the tethered anchor 100 where the end portion 106 is inserted through each of the crossing apertures of the anchor portion 104, and the anchor portion 104 is bent or folded to form a plurality of pleats 200. In some examples, and as shown, the number of pleats 200 formed is the same as the number of crossing apertures on the anchor portion 104. This configuration is achieved when the end portion 106 is brought through the first crossing aperture 108, such that the anchor portion 104 is folded between the first crossing aperture 108 and the second crossing aperture 110 and the end portion 106 passes through the second crossing aperture 110. These steps are repeated until end portion 106 passes through all the crossing apertures, as shown in FIG. 2, to form a plurality of pleats 200 in the anchor portion 104, e.g., a first pleat 202, a second pleat 204, a third pleat 206, a fourth pleat 208, and a fifth pleat 210. The pleats 200 resemble a zigzag, or accordion shape when seen from the side. This arrangement allows the anchor portion 104 to initially take on an elongate, more linear profile and then upon tensioning the tether portion 102 the anchor portion 104 folds down to an enlarged, transverse profile relative to the longitudinal axis. In this manner the anchor portion 104 can be initially deployed in a lower profile, or delivery configuration (e.g., by being inserted through a tissue structure) and subsequently tensioned down to an enlarged profile, or anchoring configuration.

FIG. 3 shows the final product in an enlarged profile or anchoring configuration after the end portion 106 is pulled away from the anchor portion 104. As shown, the pleats 200 are collapsed onto one another after tensioning the tether portion 102 to form an anchor 300 with a greater thickness than that of the anchor portion 104 in its original form shown in FIG. 1. The anchor portion 104 is collapsed in a collapsed configuration as shown in FIG. 3.

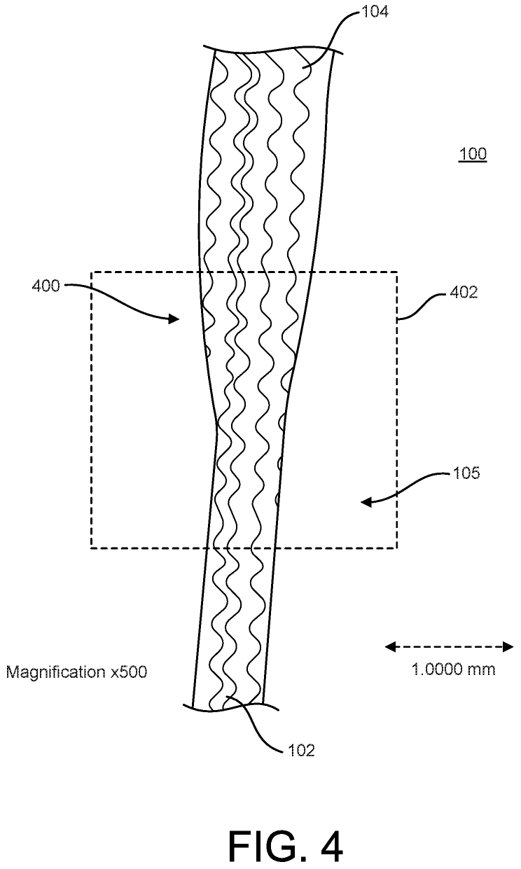

FIG. 4 shows a magnified view of the distal section 105 of the tether portion 102 and a proximal section 400 of the anchor portion 104, where the distal section 105 and the proximal section 400 combine to define a transition region 402 between the tether portion 102 and the anchor portion 104. The magnified view shows continuous braiding at the transition region 402, which varies according to the shape and structure of the tether portion 102 and the anchor portion 104. Physical properties, such as the ability for tissue ingrowth, of the tethered anchor 100 can be adjusted by changing the braiding pattern. For example, the cross-sectional shape, the pick count, and the angle of the braid are some of the properties that can be adjusted to modify performance as may relate to a use of the tether portion 102 and the anchor portion 104 (e.g. chordal repair, valve procedures, or valve annuloplasty, closing an opening or aperture formed in a wall of the heart, such as a ventricular or atrial septal wall defect).

Regardless of the component or section thereof, lowering the braid angle can increase resistance to elongation, and increasing the braid angle can decrease bending resistance as may relate to a use of the tether portion 102 and the anchor portion 104 (e.g. chordal repair, valve procedures, or valve annuloplasty, closing an opening or aperture formed in a wall of the heart, such as a ventricular or atrial septal wall defect).

The tether portion 102 and the anchor portion 104 may have various braid angles, such as 5°, 10°, 20°, 30°, 45°, 60°, any value or range between any of the foregoing examples, or more than 60°. In turn, increasing the pick count can help increases abrasion resistance. The tether portion 102 and the anchor portion 104 may have various pick counts, such as 50 picks per inch (p.p.i.), 100 p.p.i., 150 p.p.i., 200 p.p.i., 300 p.p.i., any value or range between any of the foregoing examples, or more than 300 p.p.i. In some examples, a round cross-section is preferred for the tether portion 102 to avoid the need to orient the tether portion 102 relative to a component to which it is attached (e.g., a medical device) and to reduce wear concomitant wear with any such device.

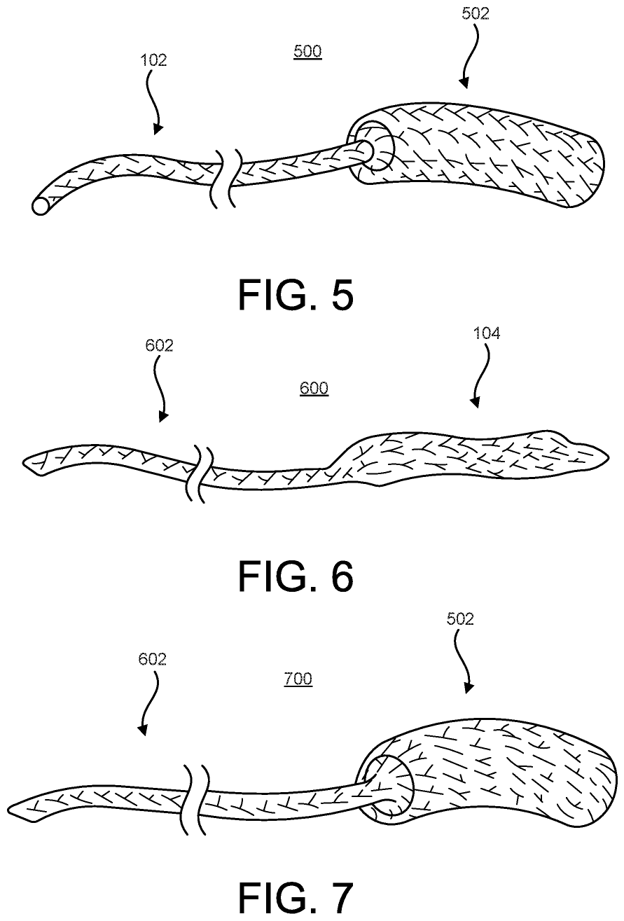

In some examples, the transition region 400 is configured to transition or expand from an aspect ratio having a rounded transverse profile to a different aspect ratio having a flat transverse profile. In one aspect of this example, a round fiber with a diameter of 0.015 inch (0.381 mm) transitions to a beaver-tail shape with a width 0.050 inch (1.27 mm). In another aspect, the beaver-tail shape has a width of 0.100 inch (2.54 mm). It is to be understood that the width of the beaver-tail shape is the distance between two ends of the anchor portion 104 measured perpendicularly with respect of the axial direction of the tether portion 102. The beaver-tail shape may have any of a variety of widths, such as 1 mm, 1.5 mm, 2 mm, 3 mm, 5 mm, any value or range between any of the foregoing examples, or more than 5 mm. In one example, FIG. 5 shows a tethered anchor 500 with the tether portion 102 having an aspect ratio close to 1:1, for example, thereby having a rounded transverse profile, and an anchor portion 502 also having the same or similar aspect ratio. In another example, FIG. 6 shows a tethered anchor 600 with a tether portion 602 having an aspect ratio greater than 10:1, for example, thereby having a flat transverse profile, and the anchor portion 104 also having the flat transverse profile. In yet another example, FIG. 7 shows a tethered anchor 700 with the tether portion 602 having the flat transverse profile and the anchor portion 502 having the round transverse profile. Other combinations, such as the tether portion and/or the anchor portion having both the round and flat transverse profiles in various sections thereof are also contemplated. Other aspect ratios may be employed, such as between 1:2 and 2:1 for the rounded transverse profile, and greater than 15:1, 20:1, or 25:1, for example, for the flat transverse profile.

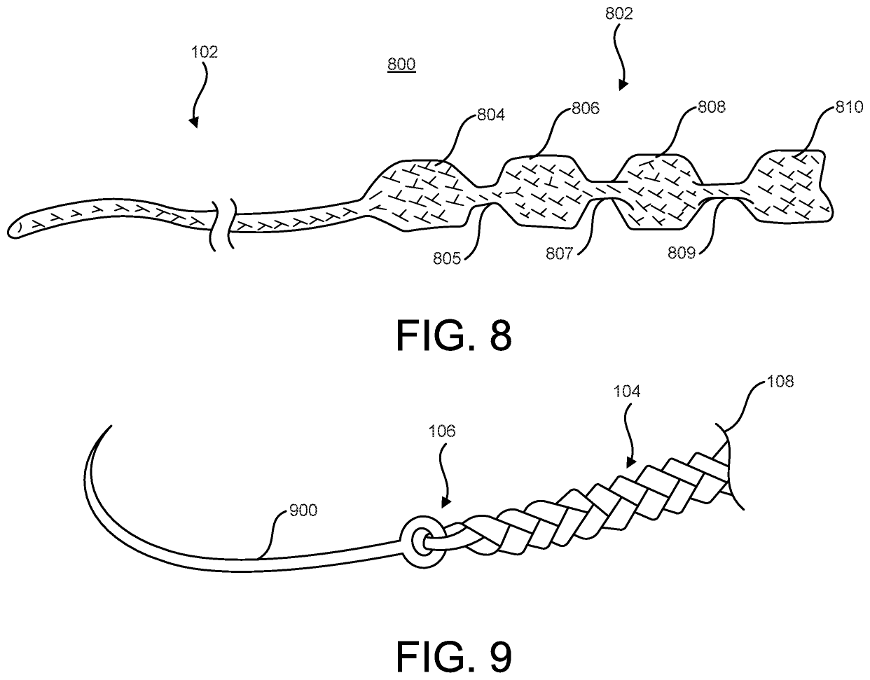

FIG. 8 shows a tethered anchor 800 according to another embodiment which includes a plurality of anchor portions 802. Although FIG. 8 shows four anchor portions 804, 806, 808, and 810, any suitable number of anchor portions can be implemented. Each pair of neighboring anchor portions 802 is connected with a connecting member such that anchor portions 804 and 806 are connected with a connecting member 805, anchor portions 806 and 808 are connected with a connecting member 807 and anchor portions 808 and 810 are connected with a connecting member 809. The number of connecting members is one fewer than the number of anchor portions 802. Similar to the previously mentioned embodiments, the anchor portions 802 fold onto one another to form an anchor similar to the anchor 300 in FIG. 3.

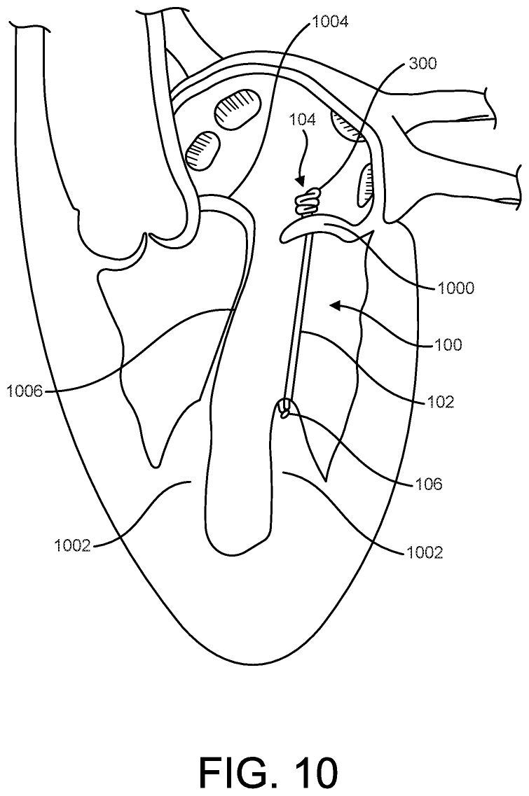

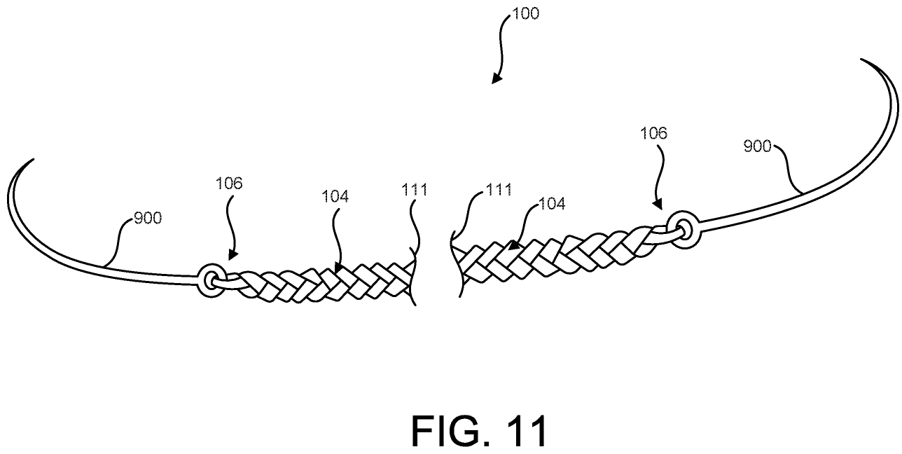

FIG. 9 shows one embodiment of an anchor portion 104 of a tethered anchor 100 having a needle 900. In this embodiment, an end portion 106 includes the needle 900 threaded onto the end portion 106 of the anchor portion 104 such that the needle 900 is implemented into the end portion 106 as part of the tethered anchor 100. In certain instances, the needle 900 is coupled, attached, or adhered to the end portion 106. A tether portion 102, as described in detail above, may be attached or coupled to an opposing end portion 106 of the anchor portion 104. As shown in FIG. 11, multiple needles 900 may be coupled to a tethered anchor. In some examples, the needle 900 can be used for skin closure, suturing soft tissue with minimal trauma, or other microsurgical procedures. The needle 900 may have a pointed edge that pierces the tissue, and in some examples also may include a cutting blade edge that can cut open tissue during microsurgical procedures. The needle 900 may have a generally C-shaped, J-shaped, or S-shaped configuration according to some examples. Additionally, the needle 900 can be of any suitable shape (straight, curved, hooked, bent, twisted, etc. in some examples), size (shorter than 3 mm, between 3 mm and 5 mm, between 5 mm and 7 mm, or longer than 7 mm in length in some examples), and material (nitinol, stainless steel, or other types of metal in some examples) as appropriate for the surgical procedure.

Furthermore, the diameter of the fibrous material used for the braiding is adjustable. In one example, the fibrous material can be ultra-high-molecular-weight polyethylene (UHMWPE) with radiopaque filler such as tungsten powder with a small particle size (for example less than 1 micron). In another example, the fibrous material can be nylon, polyurethane, polyethylene, or silicone. In another example, the fibrous material can be made of ethylene tetrafluoroethylene (ETFE) or expanded ETFE. In some embodiments, the fibrous material may be made of other fluoropolymers. In some embodiments, the fibrous material may a bioresorbable or bioabsorbable polymer.

In certain instances, the tethered anchor 100 as described herein may be used chordal repair or replacement as shown in FIG. 10. In these such instances, an apical region of a heart is percutaneously accessed with a catheter-based device. The cardiac valve is repaired by replacing at least one chordae tendineae. The replaced chordae tendineae may include the tethered anchor 100 (including a tether portion 102 and an anchor portion 104), which can also be referred to as a tissue connector due to the tether portion 102 connecting two portions of the heart tissue. In other instances, the tether portion 102 may be wrapped about a circumference of the heart or valve annulus may be arranged within a leaflet or tissue. In certain instances, the tethered anchor 100 may slightly compresses the heart to ensure that the leaflets of the valve fully close.

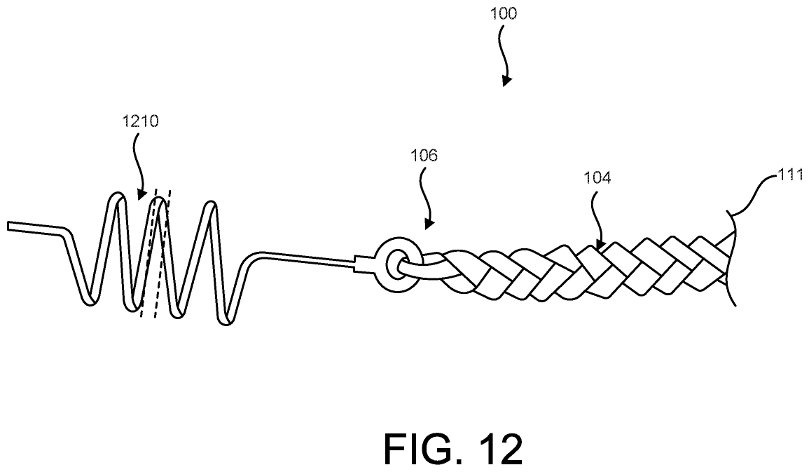

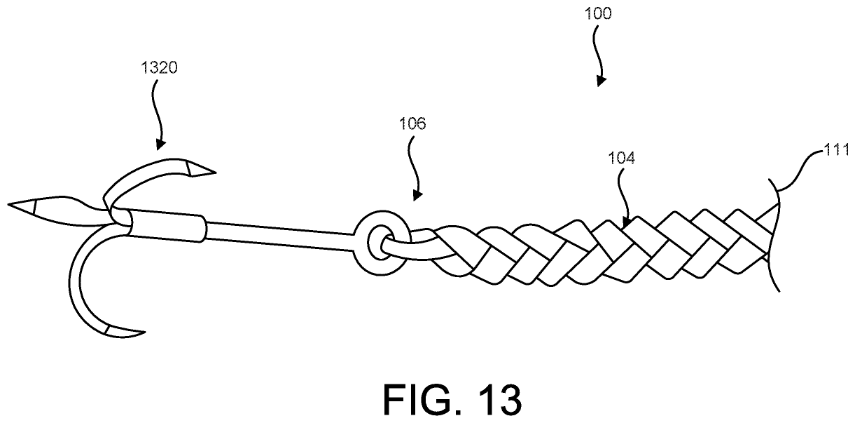

FIG. 10 shows one example of repair of a heart valve. In a healthy valve, chordae tendineae 1006 connect each leaflet 1004 to papillary muscle 1002. However, when the chordae tendinea are torn or ruptured, the tethered anchor 100 may be attached to one or more of the valve leaflets 1000 whose chordae tendineae are torn or damaged by placing or disposing the anchor portion 104 against a distal surface of the valve leaflet 1000 with respect to a papillary muscle 1002. The anchor portion 104 is bent or folded over itself to form the plurality of pleats 200 upon tensioning of the tether portion 102 as shown in FIG. 2. The tether portion 102 may be passed through each of the crossing apertures 107 in a zigzag pattern, after which the tether portion 102 is tensioned to fully collapse the pleats 200 to form the anchor 300 as shown in FIGS. 3 and 10 and the anchor 300 is collapsed to a collapsed configuration. The end portion 106 of the tether portion 102 is subsequently attached to the papillary muscle 1002 so the movement of the papillary muscle induces the movement of the valve leaflet the tethered anchor 100 used in this, or other applications, may include suture needles on both ends of the tethered anchor 100 as shown in FIG. 11 or tissue anchors on one or both ends of the tethered anchor 100 as shown in FIGS. 12-13.

FIG. 11 shows end portions of a tethered anchor 100 and needles 900 arranged at each of the ends according to some embodiments. As shown, each of the end portions 106 of the tethered anchor 100 includes needle 900 threaded onto the end portion 106 of anchor portions 104 of the tethered anchor 100. A tether portion 102, as described in detail above, may be attached or coupled to opposing ends portion 108 of the anchor portions 104. The dual needled tethered anchor 100 can be used in numerous procedures such as skin closure, suturing soft tissue with minimal trauma, or other microsurgical procedures, cardiac valve repair, or other similar procedures. In certain instances, one or both of the needles may be replaced with a tissue anchor as shown and described with reference to FIGS. 12-13.

FIG. 12 shows an end portion of a tethered anchor 100 and a tissue anchor 1210 arranged at the end portion according to some embodiments. As shown, the tissue anchor 1210 is coupled to an end portion 106 of the tethered anchor 100. The tissue anchor 1210 may be threaded onto the end portion 106 of the anchor portion 104 such that the tissue anchor 1210 is implemented into the end portion 106 as part of the tethered anchor 100. In certain instances, the tissue anchor 1210 is coupled, attached, or adhered to the end portion 106. A tether portion 102, as described in detail above, may be attached or coupled to an opposing end portion 111 of the anchor portion 104. As shown in FIG. 11, the tissue anchor 1210 may be coupled to both ends of a tethered anchor 100.

As shown in FIG. 12, the tissue anchor 1210 includes a helical shape. The tissue anchor 1210 may have one or more coils, as is shown. The number of turns or coils of the tissue anchor 1210 can be varied in order to lengthen or shorten the depth at which the tissue anchor 1210 may be arranged within a leaflet or tissue. The tissue anchor 1210 may be coupled to one or both ends of a tether 102 as discussed in detailed above with reference to FIG. 11. In addition, more than one tissue anchor 1210 may be arranged at one or both ends of the tethered anchor 100 or along the tethered anchor 100.

FIG. 13 shows an end portion of a tethered anchor 100 and a tissue anchor 1320 arranged at the end portion according to some embodiments. As shown, the tissue anchor 1320 is coupled to an end portion 106 of the tethered anchor 100. The tissue anchor 1320 may be threaded onto the end portion 106 of the anchor portion 104 such that the tissue anchor 1320 is implemented into the end portion 106 as part of the tethered anchor 100. In certain instances, the tissue anchor 1320 is coupled, attached, or adhered to the end portion 106. A tether portion 102, as described in detail above, may be attached or coupled to an opposing end portion 111 of the anchor portion 104. As shown in FIG. 11, the tissue anchor 1320 may be coupled to both ends of a tethered anchor 100. In addition, more than one tissue anchor 1320 may be arranged at one or both ends of the tethered anchor 100 or along the tethered anchor 100.

As shown in FIG. 13, the tissue anchor 1320 includes multiple barbs that are configured to embed within tissue. The tissue anchor 1320 may include three, four, five, six, or any additional number of barbs to facilitate anchoring within tissue. The tissue anchor 1320 may be coupled to one or both ends of a tether 102 as discussed in detailed above with reference to FIG. 11.

Another example of an application of the tethered anchor is in valve annuloplasty procedures in a heart, for example, where a ring around the valve in the heart (annulus) widens and changes from its normal shape. A tethered anchor 100 may be arranged to tighten or reinforce the annulus of the valve. This may prevent leakage of blood through the widened valve. The tethered anchor as described herein can be used such that the annuloplasty devices remain secured to the annulus and continue to assist in restoring the normal function of the valve.

Another example use of the tethered anchor 100 an application is in closing an opening or aperture formed in a wall of the heart, such as a ventricular or atrial septal wall defect. The tethered anchor 100 can be used to help close the opening from within the heart at the inner side of the heart wall, such that the flow of blood through the heart does not cause the anchor to detach from the wall through prolonged use due to the constant pressure exerted from within the heart.

Table 1 below shows an example of fiber diameter, braid angle, and pick count for various sections of the tether portion 102, the transition region 402, and the anchor portion 104 according to one embodiment.

TABLE 1
Fiber diameter, braid angle, and pick count
for various sections of th tethered anchor.

| | Tether portion 102 | | | | | | | | |
|---|---|---|---|---|---|---|---|---|---|
| | Proximal | | Intermediate | | Distal | | Transition | Anchor | |
| | section 101 | | section 103 | | section 105 | | region 402 | portion 104 | |
| Fiber | 0.015 | in | 0.015 | in | 0.015 | in | 0.015 in | 0.015 | in |
| Diameter | | | | | | | | | |
| (inch) | | | | | | | | | |
| Braid Angle | 15° | | 15° | | 15° | | 15° to 45° | 45° | |
| (degree) | | | | | | | | | |
| Pick Count | 50 | p.p.i. | 50 | p.p.i. | 100 | p.p.i. | 100 p.p.i. to | 150 | p.p.i. |
| (picks per | | | | | | | 150 p.p.i. | | |
| inch) | | | | | | | | | |

In some examples, the pick count varies among the different sections 101, 103, 105 of the tether portion 102. For example, the distal section 105 may have a higher pick count than the proximal section 101. In some examples, the pick count varies between the tether portion 102 and the anchor portion 104. For example, the anchor portion 104 may have a higher pick count than the tether portion 102. Because in some instances the anchor portion 104 must remain inside the heart for a prolonged period of time without detaching therefrom, it may be beneficial to have the anchor portion 104 be more resistant to abrasion. As such, a higher pick count in the anchor portion 104 may be advantageous in some instances.

In some examples, the pick count stays the same in at least one of the tether portion 102 and the anchor portion 104. In some examples, the transition region 402 and the anchor portion 104 have a larger braid angle than the tether portion 102. Generally, a larger braid angle results in reduced resistance to bending, therefore the transition region 402 and the anchor portion 104, which undergoes more folding and bending than the tether portion 102, may benefit from the larger braid angle.

According to various examples, the material of the anchored tether may include a fluoropolymer, including without limitation, polytetrafluoroethylene (PTFE) and/or expanded polytetrafluoroethylene (ePTFE), nylon, polypropylene, polyester, PVDF, silk, or other similar materials. In some examples, the anchored tether comprise a membrane, such as ePTFE, that is combined with an elastomer or elastomeric material, such as a fluoroelastomer, to form a composite material, as disclosed herein. It will be appreciated that while various examples are discussed with regard to anchored tether, the various examples and embodiments discussed herein may be universally applied across each of the anchored tethers and/or the various components of the anchored tethers discussed herein.

In some examples, echogenicity is a factor to be considered when implementing the tethered anchor such that the anchor can be accurately captured during medical imaging such as medical ultrasonography. For example, a material is more echogenic if there is hyperechoic air implemented into the material, and the material is more capable of capturing such hyperechoic air if the material comprises a hydrophobic water-immiscible matrix. In one example, the tethered anchor is made of a hydrophobic material and/or is imbibed or coated with a layer of hydrophobic agent to prevent the hyperechoic air from escaping to the environment. In another example, the tethered anchor has radiopaque fillers such as tungsten powder with a small particle size (such as less than 1 micron) such that the radiopaque fillers do not interfere with the function of the tethered anchor but allows for the tethered anchor to be visible under fluoroscopy or X-ray.

Various features have been specifically described in association with some examples and not in association with others. It is not the intent, however, to preclude the combination of features between examples. Instead, such combinations are specifically contemplated and form a part of this disclosure. The inventive concepts of this disclosure have been described both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A tethered anchor comprising:
a tether portion and an anchor portion that are continuously braided with one another;
the tether portion being elongate and extending continuously from the anchor portion; and
the anchor portion having a greater width than a width of the tether portion when the anchor portion is collapsed.

2. The tethered anchor of claim 1, wherein the tether portion defines a rounded transverse profile and the anchor portion defines a flat transverse profile.

3. The tethered anchor of claim 1, wherein the tether portion defines a flat transverse profile and the anchor portion defines a rounded transverse profile.

4. The tethered anchor of claim 1, wherein the tether portion and the anchor portion both define a rounded transverse profile.

5. The tethered anchor of claim 1, wherein the tether portion and the anchor portion both define a flat transverse profile.

6. The tethered anchor of claim 1, wherein the anchor portion defines a plurality of crossing apertures, and further wherein a length of the tether portion is passed through the crossing apertures of the anchor portion.

7. The tethered anchor of claim 1, wherein the tether portion is characterized by a first pick count and the anchor portion is characterized by a second pick count that is different than the first pick count.

8. The tethered anchor of claim 1, wherein a pick count of the tether portion and anchor portion varies.

9. The tethered anchor of claim 1, wherein a section of the tether portion is characterized by a first pick count and another section of the tether portion is characterized by a second pick count that is greater than the first pick count.

10. The tethered anchor of claim 1, wherein at least part of the anchor portion is characterized by a braid pattern configured to encourage tissue ingrowth.

11. The tethered anchor of claim 1, wherein the anchor portion comprises a plurality of individual braid filaments and the anchor portion is thermally treated to bond the individual braid filaments together.

12. The tethered anchor of claim 1, further comprising a stop component coupled to the tether portion and configured to prevent the tether portion from passing through the anchor portion.

13. The tethered anchor of claim 1, wherein at least one of the tether portion and the anchor portion incorporates an elastomeric axial member therein.

14.-35. (canceled)

36. A tethered anchor comprising:
a tether portion and at least one anchor portion that are continuously braided with one another, the tether portion being elongate and extending continuously from the at least one anchor portion and the at least one anchor portion having a greater width than a width of the tether portion; and
at least one surgical needle coupled to the at least one anchor portion.

37. The tethered anchor of claim 36, wherein the at least one anchor portion includes a first anchor portion arranged at an end of the tether portion and a second anchor portion arranged at another end of the tether portion.

38. The tethered anchor of claim 37, wherein at least one surgical needle includes a first surgical needle coupled to the first anchor portion and a second surgical needle coupled to the second anchor portion.

39. A tethered anchor comprising:
a tether portion and at least one anchor portion that are continuously braided with one another, the tether portion being elongate and extending continuously from the at least one anchor portion and the at least one anchor portion having a greater width than a width of the tether portion; and
at least one tissue anchor coupled to the at least one anchor portion.

40. The tethered anchor of claim 39, wherein the at least one anchor portion includes a first anchor portion arranged at an end of the tether portion and a second anchor portion arranged at another end of the tether portion.

41. The tethered anchor of claim 40, wherein at least one tissue anchor includes a first tissue anchor coupled to the first anchor portion and a second tissue anchor coupled to the second anchor portion
